(19) United States
(12) Patent Application Publication
(10) Pub. No.: US 2018/0296595 A1
(43) Pub. Date: Oct. 18, 2018

(54) METHODS AND COMPOSITIONS FOR CANCER TREATMENT

(71) Applicants: Memorial Sloan Kettering Cancer Center, New York, NY (US); Weill Medical College of Cornell University, New York, NY (US)

(72) Inventors: Jan GRIMM, Larchmont, NY (US); Monica L. GUZMAN, New York, NY (US); Charalambos KAITTANIS, New York, NY (US)

(73) Assignees: Memorial Sloan Kettering Cancer Center, New York, NY (US); Weill Medical College of Cornell University, New York, NY (US)

(21) Appl. No.: 15/759,161

(22) PCT Filed: Sep. 9, 2016
(86) PCT No.: PCT/US2016/050891

§ 371 Date: Mar. 9, 2018

Related U.S. Application Data
(60) Provisional application No. 62/217,449, filed on Sep. 11, 2015.

(51) Int. Cl. A61K 33/26; A61K 9/51; A61P 35/02

(52) U.S. Cl. CPC A61K 33/26; A61P 35/02; A61K 9/5161

(57) ABSTRACT

A method for treating leukemia is disclosed. The method comprises administering to a subject with leukemia an effective amount of a pharmaceutical composition comprising a naked iron oxide nanoparticle, wherein the composition is not loaded with an active agent. Also disclosed is a method for treating cancer. The method comprises administering an effective amount of a pharmaceutical composition comprising a metal oxide nanoparticle comprising a polymeric coating over a metal oxide core, wherein the nanoparticle is loaded with a reactive oxygen species (ROS)-inducing agent.

This application claims priority to U.S. Patent Application Ser. No. 62/217,449, filed Sep. 11, 2015. The entirety of the aforementioned application is incorporated herein by reference.

This invention was made with government support under Grant No. W81XWH-12-1-0509 awarded by the Department of Defense. The government has certain rights in the invention.

### FIELD

The present application relates generally to the use of metal oxide nanoparticles for cancer treatment, in particular the use of iron oxide nanoparticles as therapeutic agents for treatment of leukemia, such as acute myelogenous leukemia (AML).

### BACKGROUND

AML is often fatal. Though many patients achieve complete remission with standard chemotherapy, most relapse and die. Chemotherapy (or bone marrow transplantation) causes severe side effects. AML relapse is often attributed to the inability of drugs to target leukemia stem cells (LSCs). LSCs are refractory to current chemotherapy as they are mostly quiescent and protected by the bone marrow niche from where they are re-initiating disease. Many potent agents against LSC have poor solubility and bioavailability resulting in obstacles to clinical translation and side effects. Due to this gap in therapy surviving LSCs are able to initiate recurrence. Accordingly, there is a need for compositions and therapies that are better able to selectively target cancer cells, including cancer stem cells, such as LSCs so as to provide improved outcomes.

### SUMMARY

One aspect of the present application relates to a method for treating leukemia comprises administering to a subject with leukemia an effective amount of a pharmaceutical composition comprising a naked iron oxide nanoparticle, wherein the composition is not loaded with an active agent. In a particular embodiment, the nanoparticle further comprises a polymeric coating over the iron oxide core.

In one embodiment for leukemia treatment, the iron oxide nanoparticle includes a polymeric coating over the iron oxide core in which the nanoparticle is loaded with a reactive oxygen species (ROS)-inducing agent. In some embodiments, the reactive oxygen species (ROS) generating agent selected from the group consisting of A-type proanthocyanidins (A-PACs), erastin, bortezomib, parthenolide, elesclomol, and combinations thereof.

In certain preferred embodiments, the iron oxide nanoparticle (naked or otherwise) comprises non-stoichiometric magnetite (superparamagnetic iron oxide) with or without a coating comprising polyglucose sorbitol carboxymethylether or carboxymethyl dextran. In a particular embodiment, the iron oxide nanoparticle comprises ferumoxytol.

Exemplary leukemias for treatment include acute myelogenous leukemia, chronic myelogenous leukemia, acute lymphoblastic leukemia, chronic lymphocytic leukemia, hairy cell leukemia, T-cell prolymphocytic leukemia, large granular lymphocytic leukemia, and adult T-cell leukemia.

In another aspect, a method of treating cancer comprises administering to a subject with cancer an effective amount of a pharmaceutical composition comprising a naked metal oxide nanoparticle, wherein the composition is not loaded with an active agent. In a particular embodiment, the nanoparticle further comprises a polymeric coating over the metal oxide core.

In one embodiment for cancer treatment, the iron oxide nanoparticle includes a polymeric coating over the metal oxide core in which the nanoparticle is loaded with a reactive oxygen species (ROS)-inducing agent. In some embodiments, the reactive oxygen species (ROS) generating agent selected from the group consisting of A-type proanthocyanidins (A-PACs), erastin, bortezomib, parthenolide, elesclomol, and combinations thereof.

In certain preferred embodiments, the metal oxide nanoparticle (naked or otherwise) comprises non-stoichiometric magnetite (superparamagnetic iron oxide) with or without a coating comprising polyglucose sorbitol carboxymethylether or carboxymethyl dextran. In a particular embodiment, the metal oxide nanoparticle comprises ferumoxytol.

Exemplary cancers for treatment include solid cancers, such as prostate cancer, breast cancer, renal cell carcinoma, lung cancer, ovarian cancer, colon cancer, cervical cancer, brain cancer, liver cancer, pancreatic cancer, kidney and stomach cancer and non-solid cancers, such as leukemia, myeloma and lymphoma.

In another aspect, a pharmaceutical composition for treating cancer includes (1) a nanoparticle comprising a polymeric coating over a metal oxide core, wherein the nanoparticle is loaded with a ROS-generating agent selected from the group consisting of A-PACs, erastin, bortezomib, parthenolide and elesclomol; and (2) a pharmaceutically acceptable carrier. In one embodiment, the metal oxide nanoparticle comprises non-stoichiometric magnetite (superparamagnetic iron oxide) with or without a polymeric coating comprising polyglucose sorbitol carboxymethylether or carboxymethyl dextran.

### BRIEF DESCRIPTION OF THE DRAWINGS

The above and other objects and advantages of the application will be apparent upon consideration of the following detailed description, taken in conjunction with the accompanying figures and paragraphs.

FIG. 1 shows differences in iron homeostasis between normal cells, which maintain relatively low levels of intracellular iron, and cancer cells, which have low levels of the iron exporter enzyme, ferroportin, resulting in the trapping of iron in cells, which leads to increased ROS, thereby potentiating drug-induced oxidative stress and resultant cell death in cancer cells, while reducing cell death in normal cells. Hepcidin is a peptide hormone known to be the key regulator of iron homeostasis, affecting entry of iron into the circulation of mammals. Hepcidin binds the only known cellular iron exporter, ferroportin (Fpn; SLC40A1), leading to its internalization and degradation. As a result, active ferroportin levels are reduced, further trapping iron in cells, and consequently increasing ROS levels within.

FIG. 2, panel A shows that FH retains hydrophobic drugs at physiological pH (pH 7.4) to release them at mildly acidic conditions. FIG. 2, panel B shows that increased concentrations of drug (i.e., Flutaxl) produced increased number of FH-loaded drug molecules. FIG. 2, panel C shows that drug release occurred at mildly acidic pH, as evidenced by the degree of FH fluorescence. FIG. 2, panel D shows that FH significantly improved the efficacy of drugs by one log. FIG. 2, panel E shows that FH oxidizes substrates via a peroxidase-like activity in a dose dependent pattern; and is able to augment the effect of the ROS-generating drug bortezomib, which can be prevented by the ROS scavenger ascorbate (FIG. 2, panel F).

FIG. 3, panel A shows CD34⁺ AML LSCs possess higher basal levels of ROS that normal CD34⁺ cells. DCF was used to evaluate ROS levels by flow cytometry, whereby the left bar shows CD34⁺ AML cells, and the right bar shows CD34⁺ normal cells. Low ferroportin (SLC40A1) expression in AML patients correlates with poor prognosis in a 261 AML sample data set from two independent cohorts (GEO accession #GSE6891; Verhaak et al. 2009) (FIG. 3, panel B). FIG. 3, panel C shows FPN gene expression for primary samples (CD34+ cells) and AML cell lines relative to normal CD34+ CB cells. Each dot represents an individual sample Ferroportin is decreased up to 4-fold. FIG. 43, panel D shows representative flow cytometry histogram for the expression of FPN in CD34+ cells from a normal BM and an AML patient sample. FIG. 3, panel E shows protein expression (evaluated by flow cytometry) of FPN in primary AML patients. Blasts, progenitors (CD34+) and stem (CD34+CD38−) populations are shown. Each dot represents an individual sample.

FIG. 4 shows AML samples with low ferroportin (FPNlow) have higher levels of HMOX1 and are sensitive to FH. FIG. 4, panel A shows Gene expression for HMOX1 for FPNlow and FPNhigh samples relative to normal CD34+ from cord blood (CB). FIG. 4, panel B shows FPNlow and FPNhigh samples were treated with FH for 48 h. Cell number (top) and iron uptake (bottom) was evaluated. FIG. 4, panel C shows treatment with FH in vitro resulted in decreased survival of SLC40A1 (ferroportin) low AML CD34+ cells. FIG. 4, panel D shows Xenografts for FPNlow, or FPNhigh AML samples (the altter are rare) and CB sample were evaluated for response to FH treatment. The percent of remaining human leukemic cells and LSC (in FIG. 4, panel E) after FH treated mice relative to vehicle control therapy is shown. Each symbol represents and individual mouse error bars represent the SEM. Significance calculated by One-way ANOVA. FIG. 4, panel F shows gene expression for HMOX1 in human cells isolated from the treated mice demosntrates upregulation of HMOX-1 in response to the increased oxidatuve stress. Fold change relative to untreated.

FIG. 5 shows the in vitro viability of lymphocytes (FIG. 5, panel a), AML CD34+ (FIG. 5, panel b), leukemic blasts (FIG. 5, panel c) or LSCs (FIG. 5, panel d) from four AML patient samples (AML1-AML4) after treatment with FH nanoparticles loaded with A-PAC, a natural ROS-inducing compound.

FIG. 6 shows that dose-dependent therapeutic effects were observed in LNCaP xenografts treated with FH-bortezomib and FH alone, but not bortezomib alone or vehicle (FIG. 6, panel A and panel B). Each of the photographs in FIG. 6, panel C and panel D depicts a representative animal treated with vehicle (FIG. 6, panel C) or bortezomib-loaded FH (1.0 mM) (FIG. 6, panel D).

FIG. 7 shows results of prostate cancer therapy with FH. FIG. 7, panel A shows that prostate cancer patients with lower FPN levels (relative mRNA expression levels) have a significantly poorer prognosis. FIG. 7, panel B shows that different PC cell lines express different levels of FPN. FIG. 7, panel C shows that Western blot and FACS analysis confirms the expression data. FIG. 7, panel D shows that treating 22RV1 and DU145 with FH increased ROS in DU145, which has lower FPN levels. FIG. 7, panel E shows that adding FH to increasing amounts of erastin improves therapy efficacy more in cells with lower FPN (DU145) than in those with higher (22RV1), who need higher amounts of FH to be killed. FIG. 7, panel F shows that treatment with FH alone is more efficient in DU145 cells with low FPN (y-axis indicates absolute viability stain). FIG. 7, panel G shows that therapy of 22RV1 cells with hepcidin decreases the FP expression.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION

Some modes for carrying out the present invention are presented in terms of its exemplary embodiments, herein discussed below. However, the present invention is not limited to the described embodiment and a person skilled in the art will appreciate that many other embodiments of the present invention are possible without deviating from the basic concept of the present invention, and that any such work around will also fall under scope of this application. It is envisioned that other styles and configurations of the present invention can be easily incorporated into the teachings of the present invention, and only one particular configuration shall be shown and described for purposes of clarity and disclosure and not by way of limitation of scope.

Headings used herein are for organizational purposes only and are not meant to be used to limit the scope of the description or the enclosed paragraphs. As used throughout this application, the word “may” is used in a permissive sense (i.e., meaning having the potential to), rather than the mandatory sense (i.e., meaning must). The terms “a” and “an” herein do not denote a limitation of quantity, but rather denote the presence of at least one of the referenced items. Definitions

As used herein, the term “nanoparticle” or “NP” refers to a solid particle having a structure including at least one region or characteristic dimension with a dimension of between 1-500 nm and having any suitable shape, e.g., a rectangle, a circle, a sphere, a cube, an ellipse, or other regular or irregular shape. Non-limiting examples of nanoparticles may include liposomes, poloxamers, microemulsions, micelles, dendrimers and other phospholipid-containing systems, and perfluorocarbon nanoparticles. The term “nanoparticle” can include nanospheres, nanorods, nanoshells, and nanoprisms and these nanoparticles can be part of a nanonetwork. Without limitations, the nanoparticles used herein can be any nanoparticle available in the art or available to one of skill in the art.

The term “naked metal oxide nanoparticle” or “naked iron oxide nanoparticle” refers to a nanoparticle that is not coated with, attached (either covalently or non-covalently) to, or otherwise physically associate with, any therapeutic agents or active agents apart from the nanoparticle itself

The terms “(super)paramagnetic iron oxide nanoparticle”, “IONP” and “iron oxide nanoparticle” are used interchangeably with reference to a nanoparticle containing an “iron oxide nanoparticle core” of magnetite (Fe₃O₄) and/or maghemite (y Fe₂O₃). The IONP may further include a polymeric coating for loading drugs, and typically has a size ranging from 10 to 250 nm.

As used herein, “coated IONP” refers to an IONP with an outer shell capable of incorporating or intercalating a cargo of the present inventions, for example, a polymer coated IONP, a poly(acrylic acid)-coated IONP, an aminated IONP, a carbohydrate coated IONP, etc.

With reference to a coated nanoparticle, the terms “coating”, “outer shell”, “outer coating” or “coated” are used with to a coating on the outer surface of the nanoparticle for accommodating loaded drugs and/or targeting molecules and/or enhancing the pharmacokinetic properties of the NP or IONP. Exemplary coatings generally include, but are not limited to, carbohydrates, such as polyglucose sorbitol carboxymethylether, amides, polymers, such as poly(acrylic acid), dextran, derivatives of dextran, such as carboxymethyl dextran, a modified dextran, polyglucose sorbitol carboxymethylether, etc. Coating molecules may have functional groups, including hydroxyl, amine, forming aminated coatings, carboxyl and methyl groups, among others.

The terms “therapeutic agent” and “active agent” refer to a molecule that can be used to reduce the rate of cancer cell growth or to induce or mediate the death (e.g., necrosis or apoptosis) of cancer cells in a subject (e.g., a human). In non-limiting examples, the therapeutic agent or active agent can be a small molecule, a protein (e.g., an antibody, an antigen-binding fragment of an antibody, or a derivative or conjugate thereof), a nucleic acid, or any combination thereof. Non-limiting examples of therapeutic agents include: cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, azacitidine, axathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

As used herein, the term “loading” refers to the action of coating, attaching (either covalently or non-covalently) to, or otherwise physically associating NPs or iron oxide nanoparticles (IONPs) with a cargo molecule or a combination of cargo molecules. In some cases, cargo molecules are non-covalently attached to, or retained by, the IONPs via weak electrostatic interactions or similar weak attractive forces or other such interactions between IONPs and the cargo molecules.

As used herein, “IONPs not loaded with a therapeutic” refers to IONPs that are not coated with, attached (either covalently or non-covalently) to, or otherwise physically associate with, a therapeutic.

As used herein, “Feraheme®” (FH) or “ferumoxytol” refer to an IONP in the form of an iron oxide nanoparticle coated with a carbohydrate shell, i.e., carboxymethyl dextran or polyglucose sorbitol carboxymethylether. Feraheme® particles have an exemplary size range between 17-31 nm in diameter.

As used herein, “treatment” and “treating” refer to administration or application of a therapeutic agent to a subject or performance of a procedure or modality on a subject for the purpose of obtaining a therapeutic benefit to a disease or health-related condition. For example, a treatment may include administration of a parenteral pharmaceutical composition comprising a coated iron oxide nanoparticle to a subject in need of cancer treatment.

As used herein, “administered” or “administering” in reference to a therapeutic or compound refers to any method of providing a therapeutic or compound (i.e., cargo) loaded coated IONP to a cell or tissue in culture or to a patient such that the drug or compound has its intended effect on the cell, tissue or patient. In other words, administration of a therapeutic to a cell, tissue, tumor, etc. refers to “drug delivery”

As used herein, “patient” or “subject” refers to a mammal or animal that is a candidate for receiving medical treatment. For example, a mammal may be a human.

As used herein, “therapeutic” or “active agent” refers to a chemical or compound with biological activity against cancer. Examples of a therapeutic or active agents include, but are not limited to small molecule drugs, organic compounds, peptides, peptidomimetics, proteins, antibodies, siRNA etc.

As used herein, “effective amount”, “therapeutically effective amount,” “biologically effective amount”, and “therapeutic amount” are used interchangeably herein to refer to an amount of a therapeutic that is sufficient to achieve a desired result, i.e., therapeutic effect, whether quantitative or qualitative. In particular, a pharmaceutically effective amount, in vivo, is that amount that results in the reduction, delay, or elimination of undesirable effects (such as pathological, clinical, biochemical and the like) in the subject.

The disclosure of ranges is intended as a continuous range including every integer value between the minimum and maximum values. Thus, recitation of ranges of values herein are merely intended to serve as a shorthand method of referring individually to each separate value falling within the range, unless otherwise indicated herein, and each separate value is incorporated into the specification as if it were individually recited herein.

A “pharmaceutically acceptable carrier” may be used to facilitate administration of the iron nanoparticles parenterally, including for example, infusion, injection or implantation; or enterally, including for example pills, tablets, or suppository.

As used herein, “therapeutic effect” in reference to administration of a therapeutic or drug in vitro refers to a change in cell or tissue viability, such as a change in growth, function, death, etc. A “therapeutic effect” in reference to administration of a therapeutic or drug in vivo refers to improved health of the subject, including but not limited to reduction of tumor growth, reduction of harmful disease symptoms, a change in immune function, a change in cell, tissue, endocrine, or organ function. In other words, a therapeutic effect results in the reduction, delay, or elimination of undesirable effects (such as pathological, clinical, biochemical and the like) in the subject.

As used herein, “cancer” is a general term for diseases that are characterized by the uncontrolled, abnormal growth of cells. Cancer cells spread locally and can intravasate and spread via the bloodstream and lymphatic system to other parts of the body as metastatic cancer. Representative cancers include solid cancers, such as prostate cancer, breast cancer, renal cell carcinoma, lung cancer, ovarian cancer, colon cancer, cervical cancer, brain cancer, liver cancer, pancreatic cancer, kidney and stomach cancer, and non-solid cancers, such as leukemia, myeloma and lymphoma.

As used herein, a “cancer stem cell” refers to a cancer cell able to reconstitute the hierarchical structure of cancer tissues, and includes the capacity to: (1) self-renew, meaning the ability of either or both of the divided daughter cells to produce cells which maintain the same capacity and the degree of differentiation as the parental cell in terms of cell lineage; and (2) differentiate into various types of cancer cells constituting a cancer cell mass, whereby (like normal stem cells) the various types of cancer cells differentiated from the cancer stem cells in a sequential manner to generate a hierarchical organization of cancer stem cells at the top in terms of cell lineage.

As used herein, “tumor” or “neoplasm” refers to an abnormal mass of tissue that results from excessive cell division that is uncontrolled and progressive. A tumor may be either benign (not cancerous) or malignant. Cancer cells may be located in a tumor. A cell that is part of a tumor is a “tumor cell.”

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise. Thus, for example, reference to “a peptide” includes “one or more” peptides or a “plurality” of such peptides. With respect to documents described in the present application, all of the teachings in any issued patent or patent application publication described in this application is expressly incorporated by reference herein.

### Metal Oxide Nanoparticle Therapy for Cancer

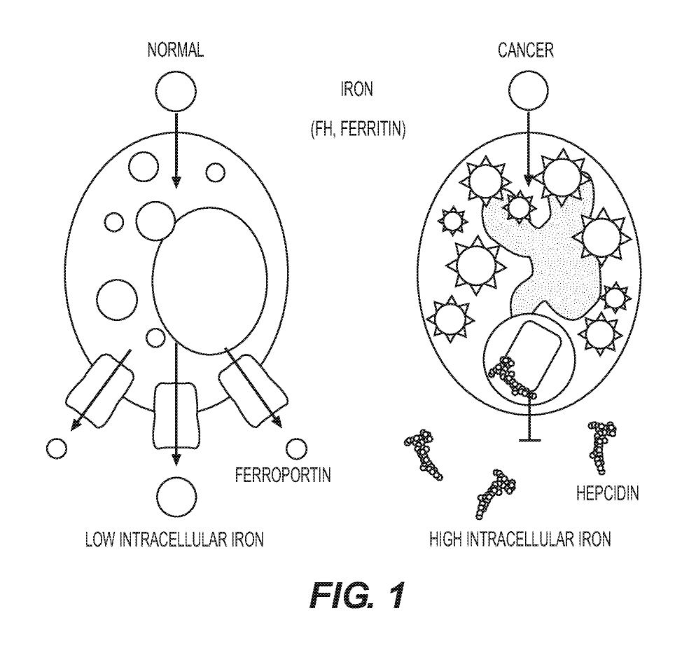

As shown in FIG. 1, normal cells are known to regulate intracellular iron concentrations through the activity of ferroportin, the sole known cellular exporter of iron. Due to the activity of this enzyme, normal cells maintain relatively low intracellular iron concentrations. Hepcidin, a key regulator of the entry of iron into the circulation in mammals, is known to regulate iron levels by inducing degradation of ferroportin, leading to accumulation (or trapping) of iron in cells.

Increased cellular iron is associated with formation of ROS, which induces cell differentiation and at higher levels, cell death. The redox-active iron in cells can catalyze damaging reactive oxygen species (ROS) via Fenton chemistry. Increased intracellular iron from FH can further lead to increased ROS, and can further potentiate drug-induced oxidative stress and resulting in iron-mediated oxidative cell death similar to ferroptosis in cancer cells.

Therefore, in one aspect, the present application provides method of treating a cancer in a subject in need thereof comprising the step of: administering to the subject an effective amount of a pharmaceutical composition comprising redox-active metal oxide nanoparticles, wherein the composition is not loaded with a chemotherapeutic agent.

In one embodiment, the redox-active metal oxide nanoparticles have dimensions in the range of 1-500 nm. In some embodiments, the redox-active metal oxide nanoparticles have dimensions in the range of 1-20 nm, 1-50 nm, 1-100 nm, 1-200 nm, 1-300 nm, 1-400 nm, 20-50 nm, 20-100 nm, 20-200 nm, 20-300 nm, 20-400 nm, 20-500 nm, 50-100 nm, 50-200 nm, 50-300 nm, 50-400 nm, 50-500 nm, 100-200 nm, 100-300 nm, 100-400 nm, 100-500 nm, 200-300 nm, 200-400 nm, 200-500 nm, 300-400 nm, 300-500 nm or 400-500 nm.

Exemplary redox active metal oxides include, but are not limited to, iron oxide, chromium oxide, gadolinium oxide, cobalt oxide, zinc oxide, nickel oxide, titanium oxide, tungsten oxide, manganese oxide, vanadium oxide, selenium oxide, molybdenum oxide, cerium oxide, and combinations thereof. Preferably, the redox-active metal oxide nanoparticle is magnetic and/or comprises an oxidation state between +1 and +9.

In a preferred embodiment, the metal oxide nanoparticle is an iron oxide nanoparticle. An iron oxide nanoparticle may comprise an iron oxide or a ferrite derived from an iron oxide. The iron oxide may be a magnetite, maghemite, or a mixture thereof. The ferrite may be an oxide of the formula M(FeₓO_{y}), where M represents any metal that forms divalent bonds, and where M represents one or more metals selected from the group consisting of Zn, Co, Mn, V, Cu, Cr, Ti, Ba, Bi, Mg, Cd and Ni.

In one embodiment, the iron oxide nanoparticle includes an iron oxide nanoparticle core” of magnetite (Fe₃O₄) and/or maghemite (γFe₂O₃). While oxidation states are typically expressed in the form of an integer (e.g., +2, +3 etc.), fractional oxidation states may be used to represent the average oxidation states of several atoms of the same element in a structure, such as magnetite, which has an average oxidation state +8/3 for iron.

In certain preferred embodiments, the metal oxide nanoparticle comprises non-stoichiometric magnetite (superparamagnetic iron oxide). In preferred embodiments, the metal oxide nanoparticle comprises non-stoichiometric magnetite coated with polyglucose sorbitol carboxymethylether, carboxymethyl dextran or other modified dextrans. In a particular preferred embodiment, the iron oxide nanoparticles are Feraheme™ (FH) or ferumoxytol (AMAG Pharmaceuticals Inc., Lexington, Mass.). FH comprises ultrasmall superparamagnetic iron oxide nanoparticles (30 mg/ml iron ion) approved by FDA for intravenous treatment of iron deficiency in patients with impaired renal function. FH has an iron oxide core and a carboxymethyl dextran coating. FH has a mean hydrodynamic diameter of 30 nm, an r₁ relaxivity of 38 s⁻¹ mM⁻¹ and an r₂ relaxivity of 83 s⁻¹ mM⁻¹ at 40 Mhz and at 37° C.

Other sources of superparamagnetic iron oxide formulations include Feridex I.V.®, an 11.2 mg/ml iron formulation (Bayer HealthCare Pharmaceuticals, Wayne, N.J.); Monofer® (iron isomaltoside 1000), an iron-carbohydrate complex formulation for intravenous administration (Pharmacosmos A/S, Holbaek, Denmark); Diafer®, a 50 mg/ml iron formulation for I.V. injection (Pharmacosmos A/S, Holbaek, Denmark); and Cosmofer, containing an iron(III)-hydroxide dextran complex (Pharmacosmos A/S, Holbaek, Denmark), all of which are approved for the treatment of iron deficiency.

In some cases, the pharmaceutical compositions may comprise non-magnetic iron oral supplements in place of, or in addition to NPs or IONPs. Iron can be orally supplemented using various pharmacological forms, including iron (II) sulfate, the most common and inexpensive salt (e.g., Feratab, Fer-Iron, Slow-FE, etc.) and in complex with gluconate, polysaccharide, dextran, fumurate, carbonyl iron, heme (e.g., Feraheme®), pyrophosphate, and other salts. Exemplary commercial oral iron formulations include, but are not limited to: Feosol; NovaFerrum 125 and NovFerrum 50; Icar Pediatric; Fesol Caplets; Ircon; Hemocyte; Ferrous Fumarate Tablets; Nephro-Fen Feostat; Ferrous Fumarate with DSS Timed capsules; Ferro-DSS Caplets; Ferro-Sequels; Fergon; Ferrous Gluconate Tablets; Ferrous Sulfate Elixer; Ferrous Sulfate SolutionI Fer-Gen-Sol Drops; Mol-Iron; Feratab; Ferrous Sulfate Tablets EC; Ferrous Sulfate Tablets; Slow FE; Ferres-150; Fe-Tinic; Hytinic; Niferes-150; Niferex-Elixer; Niferex; Enfamil Fer-In-Sol; EZFE 200; Fe-20; Femiron; Ferate; Fer-Iron; Ferretts IPS; Ferrimin 150; Good Neighbor Pharmacy Iron; Good Neighbor Pharmacy Slow Release Iron; Leader Iron Tablets; Natural Slow Release Iron; Nature's Blend Slow Iron; ProFe; Proferrin ES; Right Aid Iron; Right Aid Slow Release Iron; or Slow Fe.

In certain embodiments, the nanoparticle may further include a polymeric coating for loading ROS-inducing agents or chemotherapeutic drugs, and/or providing more favorable pharmacokinetic properties (e.g., stability in bloodstream etc.) Suitable polymeric coating materials include, but are not limited to carbohydrates, such as polyglucose sorbitol carboxymethylether, amides, polymers, such as poly(acrylic acid), dextran, dextran derivatives, such as carboxymethyl dextran, polyoxyethylene fatty acid esters, polysorbate, collagen, hyaluronic acid, chitosan, elastin, alginate, actin, gelatin, cellulose and cellulose derivatives, synthetic polymers, such as polyethylene glycol (PEG), polyethylene oxide (PEO), polycaprolactone (PCL), polylactic acid (PLA), polyglycolic acid (PGA), poly(lactic-co-glycolic) acid (PLGA), poly(3-hydroxybutyrate-co-3-hydroxyvalerate) (PHBV), polydioxanone (PDO), poly(L-lactide-co-caprolactone), poly(ester urethane) (PEU), poly(L-lactide-co-D-lactide), poly(ethylene-co-vinyl alcohol), poly(acrylic acid) (PAA), poly(vinyl alcohol) (PVA), polyvinylpyrrolidone (PVP), polystyrene (PS), polyaniline (PAN), polyacrylamide (PA), polymethylmethacrylate (PMMA), polyglutamic acid (PGA), polyalkylcyanoacrylate, oleic acid, glyceryl monooleate (GMO), Pluronic F-127, poly(amido amine), poly(ethyleneimine), and combinations thereof. Coating molecules may have or may be further modified with reactive functional groups, including hydroxyl, amine, forming aminated coatings, carboxyl and methyl groups, among others.

In one embodiments, the polymer coating has a thickness in the range of 1-100 nm. In some embodiments, the polymer coating has a thickness in the range of 1-3 nm, 1-10, nm, 1-30 nm, 3-10, nm, 3-30 nm, 3-100 nm, 10-30 nm, 10-100 nm or 30-100 nm.

In another aspect, a pharmaceutical composition for use in the disclosed methods may comprising metal nanoparticles loaded with a reactive oxygen species (ROS)-inducing agent. Such compositions may be administered to a patient with cancer alone or in conjunction with a ROS-inducing process. Alternatively, redox-active nanoparticle compositions without an added ROS-inducing agent, therapeutic agent, or both, may be administered to a patient in conjunction with a ROS-inducing process. As further shown below, the addition of an ROS-inducing agent provides selective and synergistic killing of various cancer cell targets.

ROS are molecules or ions formed by one or more unpaired electrons of oxygen. The unpaired O₂ electrons react to form partially reduced highly reactive species that are classified as free radical or non-radical oxygen species. Oxygen free radicals include superoxide anions (O2.⁻), hydroxyl radical (OH.), nitric oxide (NO.), organic radicals (R.), peroxyl radicals (ROO.), alkoxyl radicals (RO.), thiyl radicals (RS.), sulfonyl radicals (ROS.), and thiyl peroxyl radicals (RSOO.). Non-radical ROS include hydrogen peroxide (H₂O₂), delta state singlet oxygen (¹O₂), ozone (also known as trioxygen) (O₃), organic hydroperoxides (ROOH), and hypochlorous acid (HOCl).

Reactive oxygen species (ROS) play an essential role in maintaining cellular homeostasis, and levels of ROS are regulated by redox enzymes and reduced factors such as glutathione. Excess levels of ROS can result in DNA and cellular damage which can contribute to development of tumors. Cancer cells exhibit increased metabolic activity and ROS levels compared to normal cells and, with threshold limits, ROS contribute to cancer cell homeostasis and growth. However, where treatment of cancer cells with ROS-inducing agents exceeds the threshold for ROS and this results in activation of multiple cell death pathways

The induction of oxidative stress and production of ROS ultimately causes lipid peroxidation, resulting in activation of the antioxidant defense system such that natural antioxidants, including GSH, SOD, CAT, and GPx remove excess free radicals and peroxides. However, if the degree of oxidation is beyond the capacity of the body's natural antioxidant molecules, the levels of GSH, SOD, CAT, GPx, will be reduced and may eventually lead to apoptosis.

One or more ROS-inducing agents may be loaded into the metal oxide nanoparticles of the present application. Exemplary ROS-inducing agents include, but are not limited to, erastin, bortezomib, elesclomol, parthenolide, and A-type proanthocyanidins (A-PACs), and combinations thereof. Additional ROS-inducing agents include, but are not limited to, inhibitors of glutathione synthesis, inhibitors of other antioxidant enzymes, proteasome inhibitors, inhibitors of the anti-apoptotic Bcl-2 family members, and Akt or other important regulators of apoptosis. Exemplary ROS-inducing compounds further include, but are not limited to: photosensitizers, MG132 (CAS 133407-82-6, Calbiochem), bafilomycin A1, concanamycin A, dichloroacetate (DCA), other proanthocyanidins (PACs), buthionine sulfoximine, metformin, anthracyclines, adriamycin (doxorubicin), adriamycinone (doxorubicinone), daunomycin, daunomycinone, daunorubicin, and daunorubicin derivatives such as 5-iminodaunorubicin, ubiquinone, Acid Blues 25, 80, and 41, Acid Green 25, anthraquinone and its derivatives, such as 2-bromoanthraquine, 1,2-dihydroxyanthraquinone, 1,8-diaminoanthraquinone, 2,6-diaminoanthraquinone, 1,5-dichloroanthraquinone, 1,2-diaminoanthraquinone, and 2-chloro-anthraquinone, NG132, quinizarin, anthrarufin, quilalizarin, aloe-emodin and related compounds such as 5-nitro-aloe-emodin, 5-amino-aloe-emodin. 2-allylaloe-emodin, averufin, kalafungin, alizarin complexone dihydrate, quercetin dihydrate, acid black 48, procytoxid, leucotrofina, azimexon, and methoxycin-narnonitrile.

PACs comprise a class of polyphenols found in a variety of plants. Chemically, they are oligomeric flavonoids, more specifically, polymeric flavan-3-ols whose elementary units are linked by C—C and occasionally C—O—C bonds. Many are oligomers of catechin and epicatechin and their gallic acid esters. More complex polyphenols, having the same polymeric building block, form the group of tannins. The flavan-3-ol units have the typical C6-C3-C6 flavanoid skeleton. The three rings are distinguished by the letters A, B and C. They differ structurally according to the number of hydroxyl groups on both aromatic rings and the stereochemistry of the asymmetric carbons on the heterocycle. The most common interflavanol linkages are C—C bonds established between the C4 of one flavanoid unit (“extension or upper unit”). Such proanthocyanidins belong to the so-called B-type (dimeric) and C type (trimeric) proanthocyanidins. Compounds with doubly linked units (one C—C and one C—O; “A type linkage) have also been reported in some food sources such as tea leaf, cocoa and cranberry fruits. In these A-type proanthocyanidins an additional ether linkage between the C2 of the upper unit and the oxygen-bearing C7 or C5 of the lower one is formed in addition to the usual C4-C8 or C4-C6 bond.

PACs may be synthetic products or natural products. PAC compounds can be obtained in a pure and isolated form by extraction and purification from any one of a variety of botanical sources. Natural PACs, including A-type PACs, can be derived from colored plants and vegetables containing a high content of polyphenol, including cranberry, blueberry leaves, grape seeds, taro, including the skin of taro root, bilberry, elderberry, plum, blackberry, strawberry, red currant, black currant, cherry, raspberry, currant, hibiscus flower, green pepper, beans, peas, soybean skin, a red cabbage, a purple corn, a purple sweet potato, herbs, spices, fern, nuts, bark, including spices therefrom, such as cinnamon etc.

Various parts of a plant may provide a source of PACs for the present application. Useful plant starting materials include, for example, leaves, petals, calyx, flowers, leafstalks, fresh tops, roots, stems, seeds, pods, rhizomes, bark, cambiums, lumbers, mycocecidium, fruits, tree saps, resin, grape peels, apple, onion, avocado and citrus; pomas of apple, wine, grain hull, straw and hay; lumps from oily seeds derived from olive, oilseed rape and canola; and the extracts from other oily crops etc.

In some embodiments, the NPs or IONPs are subjected to a ROS-inducing process, such as radiation therapy or photodynamic therapy (PDT) to generate hydroxyl radicals for delivery or co-delivery to target cancer cells. PDT is a treatment employing a photosensitizer or photosensitizing agent, and a particular type of light. Exemplary photosensitizers include porfimer sodium, Photofrin®, 5-aminolaevulinic acid (5-ALA), 5,10,15,20-tetrakis(m-hydroxyphenyl) chlorin (Foscan), methyl aminolevulinate (Metvix), Pd-bacteriopheophorbide (Tookad), verteporfin (Visudyne), methylene blue, chlorin e6 (Ce6), 2-devinyl-2-(1-hexyloxyethyl)pyropheophorbide (HPPH), coomasie blue, and combinations thereof.

When photosensitizers are exposed to a specific wavelength of light, they produce a form of oxygen that kills nearby cells. Each photosensitizer is activated by light of a specific wavelength. This wavelength determines how far the light can travel into the body. Thus, a suitable combination of one or more specific photosensitizers in conjunction with a specific wavelength can be used to treat different areas of the body with PDT.

The photosensitizer may be incorporated into the nanoparticle and/or it may be injected into the bloodstream before administration of the nanoparticles. When injected into the bloodstream, the photosensitizer is absorbed by cells all over the body but stays in cancer cells longer than it does in normal cells. Approximately 24 to 72 hours after injection, when most of the photosensitizer has left normal cells but remains in cancer cells, the tumor is exposed to light. The photosensitizer in the tumor absorbs the light and produces an active form of oxygen that kills nearby cancer cells.

The light used for PDT can come from a laser or other sources. Laser light can be directed through fiber optic cables (thin fibers that transmit light) to deliver light to areas inside the body. For example, a fiber optic cable can be inserted through an endoscope into, for example, the lungs or esophagus to treat cancer in these organs. Other light sources include light-emitting diodes (LEDs), which may be used for surface tumors, such as skin cancer.

In some embodiments, the tumor, NPs or both are irradiated with radiation having a wavelength greater than 400 nm. More specifically, in certain exemplary embodiments the tumor, NPs or both may include the photosensitizers (i) 5-aminolaevulinic acid (5-ALA), (ii) 5,10,15,20-tetrakis(m-hydroxyphenyl) chlorin (Foscan), (iii) methyl aminolevulinate (Metvix), (iv) Pd-bacteriopheophorbide (Tookad), (v) photosensitizer concentrated distillate of hematoporphyrins (Photofrin), or (vi) verteporfin (Visudyne), where the tumor, NPs or both which is/are irradiated, respectively, at: (i) a maximum light intensity of about 630 nm; (ii) a wavelength of about 585 to about 740 nm; (iii) a wavelength of about 570 to about 670 nm; (iv) a wavelength of about 615 to about 800 nm; (v) a wavelength of about 600 to about 750 nm; (vi) a wavelength of about 450 to about 600 nm.

In some embodiments, the pharmaceutical composition further comprises NPs or IONPs that are loaded with one or more therapeutic agents. The therapeutic agent may be any small molecule chemical compound or large molecule biological macromolecule having anti-cancer activity. The one or more therapeutics may be retained by weak electrostatic interactions with the polymeric coating in the NP or IONP. Exemplary therapeutic agents with anti-cancer activity include, but are not limited to, hepcidin mini-peptide PR65 (Ramos, E. et al. (2012), Blood 120(18): 3829-36), alkylating agents, antimetabolites, anthracyclines, antitumor antibiotics, monoclonal antibodies, platinums, signaling pathway inhibitors, NF-κB inhibitors, topoisomerase inhibitors, tyrosine kinase inhibitors, photosensitizers, plant alkaloids, histone deacetylase inhibitors, methotrexate, 6-mercaptopurine or 5-fluorouracil (5FU), daunorubicin, doxorubicin, idarubicin, epirubicin, mitoxantrone, bleomycin, alemtuzumab (Campath), bevacizumab (Avastin), cetuximab (Erbitux), gemtuzumab (Mylotarg), ibritumomab (Zevalin), panitumumab (Vectibix), rituximab (Rituxan), tositumomab (Bexxar), trastuzumab (Herceptin), cisplatin, carboplatin, oxaliplatin, Vinca alkaloids, taxanes, paclitaxel, docetaxel, epipodophyllotoxins, vorinostat (suberoylanilide hydroxamic acid (SAHA), Zolinza), romidepsin, panobinostat, valproic acid, belinostat, mocetinostat, PCI-24781, entinostat, SB939, resminostat, givinostat, CUDC-101, AR-42, CHR-2845, CHR-3996, 4SC-202, sulforphane and combinations thereof.

In some embodiments, the NPs or IONPs may be further coated, conjugated to or modified with a tumor-specific or cell/tissue specific targeting agent. The targeting agent may be a small molecule (e.g., folate, adenosine, purine, lysine), peptide, ligand, antibody fragment, aptamer or synbody. Such compositions may allow for the use of a lower dose of cytotoxic drugs, reduce adverse events, increase efficacy, and reduce the possibility of the drugs being rapidly cleared from targeted tumors or cancer cells. Targeted compositions can allow for the nanoparticles and other active agents to be taken up by cancer cells so as to promote more efficient delivery so as to better promote apoptosis and limit the potential of chemoresistance and systemic toxicities.

In some embodiments, the cell targeting agent is directed to tumor associated antigen, preferably a cell surface antigen. Examples of tumor associated antigens include, but are not limited to, adenosine receptors, alpha v beta 3, aminopeptidase P, alpha-fetoprotein, cancer antigen 125, carcinoembryonic antigen, cCaveolin-1, chemokine receptors, clusterin, oncofetal antigens, CD20, epithelial tumor antigen, melanoma associated antigen, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyosinase, and tyrosine kinases. In certain preferred embodiments, the cell targeting agent is folate or a folate derivative that binds specifically to folate receptors (FRs).

The reduced folate carrier (RFC) system is a low-affinity, high capacity system that mediates the uptake of reduced folates into cancer cells at pharmacologic (μM) concentrations. The concentration of physiologic folates is in the range of 5 to 50 nM. Therefore, high affinity human FRs exist and are encoded by a family of genes whose homologous products are termed FR type α, β, γ, or δ, which are also described as FR1, FR2, FR3, or FR4, respectively. The membrane isoforms FR1, FR2, and FR4 can bind and transport folate or folate derivatives into the cell, while FR3 lacks a membrane anchor and is secreted from the cell. FR1 and FR2 bind folate and 6S 5-formyltetrahydrofolate (i.e., leucovorin) with similar yet different affinities 1.5 nM versus 0.35 nM (folate) and 800 nM versus 7 nM (leucovorin), respectively. 6S 5-methyltetrahydrofolate is the predominate folate in the blood and has similar affinities for FR1 and FR2, 55 nM and 1 nM, respectively. While PC3 human prostate cancer cells do not significantly express FR (e.g., FR1) in culture, FRs are expressed by PC3 tumors. FRs are also expressed by BrCa cells are associated with poor outcomes or transport folate via these receptors despite resistance to methotrexate.

Most nonproliferative tissues lack functional FR expression. FR expression in proliferating normal tissues is restricted to the luminal surface of certain epithelial cells and thus inaccessible to the circulation. However, the presence of high levels of FR2 (high affinity receptor) on malignant tumors and leukemias are exposed to circulation making them an attractive candidate for tumor-specific therapeutics. The kidney, where FR1 (moderate affinity receptor) is expressed in the proximal tubules, is protected from FR-targeted therapies that are excluded from glomerular filtration. Further protection is a result of the renal folate conservation mechanism where after FR-mediated endocytosis by renal tubular cells there is rapid dissociation of the folate and transport across the basolateral membranes into the blood.

In certain compositions, the targeting agent may be an antibody or peptide capable of binding tumor associated antigens consisting of put not limited to: adenosine receptors, alpha v beta 3, aminopeptidase P, alpha-fetoprotein, cancer antigen 125, carcinoembryonic antigen, caveolin-1, chemokine receptors, clusterin, oncofetal antigens, CD20, epithelial tumor antigen, melanoma associated antigen, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyosinase, tyrosine kinases, etc.

The compositions of the present application may be used to treat a variety of solid and non-solid cancers. A representative but non-limiting list of cancers that the disclosed methods can be used to treat is the following: leukemia, myeloid leukemia, acute myelogenous leukemia, lymphoma, B cell lymphoma, T cell lymphoma, mycosis fungoides, Hodgkin's Disease, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. Compositions disclosed herein may also be used for the treatment of precancer conditions such as cervical and anal dysplasias, other dysplasias, severe dysplasias, hyperplasias, atypical hyperplasias, and neoplasias.

In preferred embodiments, the subject has leukemia. Exemplary leukemias include acute myelogenous leukemia, chronic myelogenous leukemia, acute lymphoblastic leukemia, chronic lymphocytic leukemia, hairy cell leukemia, T-cell prolymphocytic leukemia, large granular lymphocytic leukemia, and adult T-cell leukemia.

Preferably, the cancer exhibits a ferroportin activity profile sensitive to treatment with FH and/or FH-loaded drugs. In some cases, the cancer cells include a small and discrete population of cancer stem cells, such as leukemia stem cells (LSCs), which possess self-renewal capacity and are responsible for the continued proliferation and propagation of bulk cancer cells. Similar to their normal counterparts, such as hematopoietic stem cells (HSCs), these cells have the capacity to recreate the entire tumor.

To determine whether cancer cells or specific cancer cell populations exhibit perturbations of ferroportin and/or hepcidin activity or expression resulting in increased sensitivity to FH other metal oxide nanoparticles, cancer cells and specific cancer cell populations, including cancer stem cells, can be isolated using suitable markers by FACS and analyzed for their levels of activity or expression of ferroportin, hepcidin protein (encoded by HAMP gene). These and other genes involved in ROS regulation may be evaluated and correlated with known genetic markers (including NPM1, FLT3, KIT, DNMT3A, TET2, IDH1/2, TP53).

To characterize ROS generation, cell permeable compounds 2′,7′-dichlorodihydrofluorescein diacetate (H2DCFDA) and hydroethidine (HE) may be utilized. H2DCFDA measures H₂O₂ generation, whereas HE detects broader peroxide generation; the use of these two dyes can facilitate further information about different types of ROS generated.

Thiol groups in protein and non-protein compounds (e.g., glutathione) participate in oxidative metabolism. To assess the effects of metal oxide NP treatment on thiols in normal and/or cancer stem cells, such as LSCs, monobromobimane may be used. The OxyIHC Oxidative Stress Detection Kit (EMD Millipore, Billerica, Mass.) may be used to stain oxidized proteins. Immunoblots and real-time RT-PCR may be used to assess differences in key components of redox regulation including heme-oxygenase-1 (HMOX-1), thioredoxin, thioredoxin reductase, Glutathione-S transferase pi 1 (GSTP1) and carbonyl reductase (CBR1). As an additional control, prior to treatment with metal oxide NPs, cells may be pre-incubated with glutathione as a ROS scavenger to buffer ROS generation and decrease the toxicity of metal oxide NPs.

Xenotransplants of human cancer cells in mice may be set up to evaluate the effects of treatment in vivo using the metal oxide NP compositions in accordance with the methods and compositions described in the present application. In certain embodiments, primary cancer cells are injected into a sub-lethally irradiated immunodeficient mouse. Examples of immunodeficient mice include but are not limited to NSG mice (NOD/SCID/γc^{-/-}; or NOD/scid IL2rγ^{null}), NOG mice (NOD/γc^{-/-} or NOD/scid/IL2rγ^{Trunc}), NOD mice (non-obese diabetic), SCID mice (severe combined immunodeficient mice), NOD/SCID mice, nude mice, BRG mice (BALB/c-Rag2^{null}/IL2rγ^{null}), Rag 1^{-/-} mice, Rag 1^{-/-}/γc^{-/-} mice, Rag 2^{-/-} mice, and Rag 2^{-/-}/γc^{-/-} mice. Three to four weeks following the injection, after engraftment of the human cells in the mouse, treatment with metal oxide NPs, with or without loaded drugs, and appropriate controls may be initiated and continued for four weeks, with therapy every 3 d day. Following this period, the mice are sacrificed and evaluated for cancer engraftment and degree of cancer cell killing compared to controls.

In certain embodiments, a two-step augmentation approach can be used where administration of an ROS-upregulating drug or ROS-inducing treatment is followed by FH administration after a pre-determined time period. In another embodiment, enzyme-mimicking nanoformulations can be used, including c-encapsulating FH or oxidase-mimicking cerium oxide nanoparticles (Asati, A. et al. (2009), Angewandte Chemie 48(13): 2308-12); Santra, S. et al. (2010), Mol. Pharm. 7(4): 1209-22).

LSCs and other cancer stem cells have unique biological properties with intricate mechanisms to promote growth and survival. Aberrant surface phenotypes, dysregulated autonomous programs for survival, apoptosis and differentiation, and interactions with the surrounding bone marrow microenvironment comprise potentially cancer cell-specific and/or cancer stem cell-specific characteristics that can be exploited for therapeutic purposes. Indeed, as further shown below, an aberrant iron metabolism with decreased ferroportin may be an important hallmark of LSC biology. Increased cellular iron is associated with formation of ROS, which induces cell differentiation and at higher levels cell death. Therefore, LSCs and other cancer cells may be particularly sensitive to drugs that increase iron and ROS, FH being a prime candidate as it increases both.

When targeting and evaluating e.g., leukemia treatments with the compositions in the present application, CD34⁺ LSC cells may be isolated with a magnetic separator from primary patient AML samples (IRB# 0909010629), followed by cell sorting using phenotypic markers specific for LSC cells (CD45dim, CD34⁺CD38⁻CD123⁺). Normal CD34⁺ bone marrow cells (HSCs) may be purchased (Stem Cell Technologies) and used to further isolate into CD34⁺CD38⁻CD123⁻ cells. For patient AML samples, cell surface marker reagents may be used to distinguish between lymphocytes, leukemic blasts, leukemic stem and progenitor cells by FACS.

Suitable marker reagents may be similarly employed to isolate and evaluate ferroportin profiles in other cancer cells or cancer stem cells. In certain embodiments, the marker reagents are positive for one or more of the cell markers Twist, Snail, Slug, Zeb1, Zeb2, vimentin, N-cadherin, E-cadherin, EpCAM, fibronectin, CD24, CD26, CD29, CD44, CD133, and CD166. In some embodiments, normal (benign) cells and malignant cells may be cultured and incubated with FH, carboxy-dextran polymer nanoparticle (CDNP) or vehicle at different doses (0.3 mg-3.0 mg) for 48 h. CDNP provide the same capabilities for drug loading and delivery but lack the iron core and therefore peroxidase function.

To define sensitivity to FH and/or FH-drugs, ROS content and protein oxidation may be evaluated using conventional methods known in the art. For example, cell viability, which provides an indicator of cell killing, may be evaluated by multiparameter flow cytometry (FACS) staining for Annexin V and 7AAD. More particularly, viability or cell killing may be characterized on the basis of the proportion of annexin V⁻/7AAD⁻ populations normalized to a vehicle control. Iron content of the cells may be evaluated using inductively coupled plasma mass spectrometry (ICP-MS). In addition, drug uptake from FH- or CDNP-loaded samples may be facilitated with the use of fluorochrome DiL; unloaded FH uptake may be determined by FACS.

### Pharmaceutical Compositions

Another aspect of the present application relates to a pharmaceutical composition for treating cancer. Any of the above-described nanoparticle compositions may be employed in a pharmaceutical composition for treating cancer. In one embodiment, the pharmaceutical composition comprises (1) metal oxide nanoparticles loaded with a ROS-generating agent selected from the group consisting of A-PACs, erastin, bortezomib, parthenolide and elesclomol; and (2) a pharmaceutically acceptable carrier. In a preferred embodiment, the metal oxide nanoparticle comprises non-stoichiometric magnetite (superparamagnetic iron oxide) with or without a polymeric coating comprising polyglucose sorbitol carboxymethylether or carboxymethyl dextran.

In some embodiments, the pharmaceutical composition of the present application is administered parenterally by injection or infusion, such as intravenous, intraarterial, intramuscular, intracardiac, subcutaneous, intraosseous, intradermal, intrathecal, intraperitoneal, intravesical injection or infusion. Other parenteral administration include transdermal, sublingual, buccal, vaginal, inhalational, epidural (i.e., peridural), and intravitreal administration.

The composition may be administered in an amount effective for killing tumor cells or reducing their growth. Dosages for administration can be determined by physical and physiological factors such as body weight, severity of condition, the type of disease being treated, previous or concurrent therapeutic interventions, idiopathy of the patient and the route of administration. The practitioner responsible for administration will, in any event, determine the concentration of active ingredient(s) in a composition and appropriate dose(s) for the individual subject.

In certain embodiments, a dose may also comprise from about 1 μg/kg/body weight, about 50 μg/kg/body weight, about 100 μg/kg/body weight, about 500 μg/kg/body weight, about 1 mg/kg/body weight, about 5 mg/kg/body weight, about 10 mg/kg/body weight, about 30 mg/kg/body weight, about 40 mg/kg/body weight, about 50 mg/kg/body weight, about 100 mg/kg/body weight, or more per administration, and any range derivable therein. In non-limiting examples of a derivable range from the numbers listed herein, a range of about 5 μg/kg/body weight to about 5 mg/kg/body weight, about 50 μg/kg/body weight to about 50 mg/kg/body weight, etc., can be administered.

In one embodiment of the application, the pharmaceutically effective amount is defined as: (i) 1-50 mg Fe/kg body weight and/or (ii) 1-10 mg Fe/ml of an administered iron nanoparticle concentration. These doses, as described herein, are considered low doses and they do not cause (direct) cytotoxic effects to the cancer or normal/healthy cells.

Pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

Below are disclosed non-limiting exemplary embodiments. Further embodiments and advantages of the application will appear from the following description taken together with the accompanying drawings.

### EXAMPLES

### Example 1

### LSCs in AML are Selectively Targeted for Cell Death

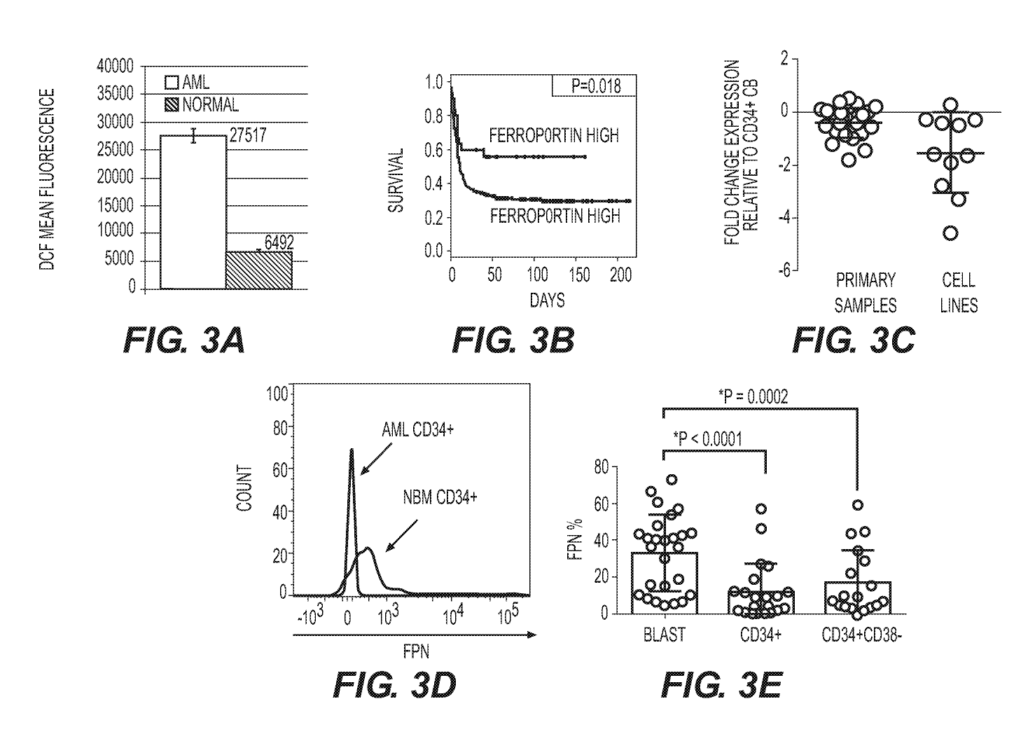

LSCs have unique biological properties with intricate mechanisms to promote growth and survival. To evaluate differences between LSCs in AML and normal bone marrow (BM) samples, levels of ferroportin (SLC40A1) were evaluated in primary AML CD34⁺ (n=4) and normal CD34⁺ bone marrow (BM) samples (n=2). As shown in FIG. 3, panel A, CD34⁺ AML LSCs possess higher basal levels of ROS that normal CD34⁺ cells.

To determine whether there is a correlation between ferroportin activity and survival in AML patients, a publicly available gene expression dataset from AML patients (GEO accession #GSE6891; Verhaak et al., Haematologica (2009) 94(1):131-4) was analyzed. The results of this analysis showed that low levels of ferroportin correlated with poor outcome (p=0.018) in AML patients (FIG. 3, panel B).

To examine whether LSCs exhibit lower ferroportin levels than normal bone marrow cells, the ferroportin levels in several AML patient samples were analyzed and compared against the ferroportin level in normal bone marrow cells. For example, an analysis of one LSC/HSC specific dataset (Majeti et al., PNAS (2009) 106(9):3396-401) showed that ferroportin levels are significantly lower in LSCs compared to their normal counterparts (p=0.008). Further experiment shows that both primary AML cells, as well as cell lines, have less expression of ferroportin than the CD34+ cells of normal cord blood (CB) (FIG. 3, panel C) or normal bone marrow (FIG. 3, panel D). Decreased levels of SLC40A1 were also observed in LSCs (FIG. 3, panel E).

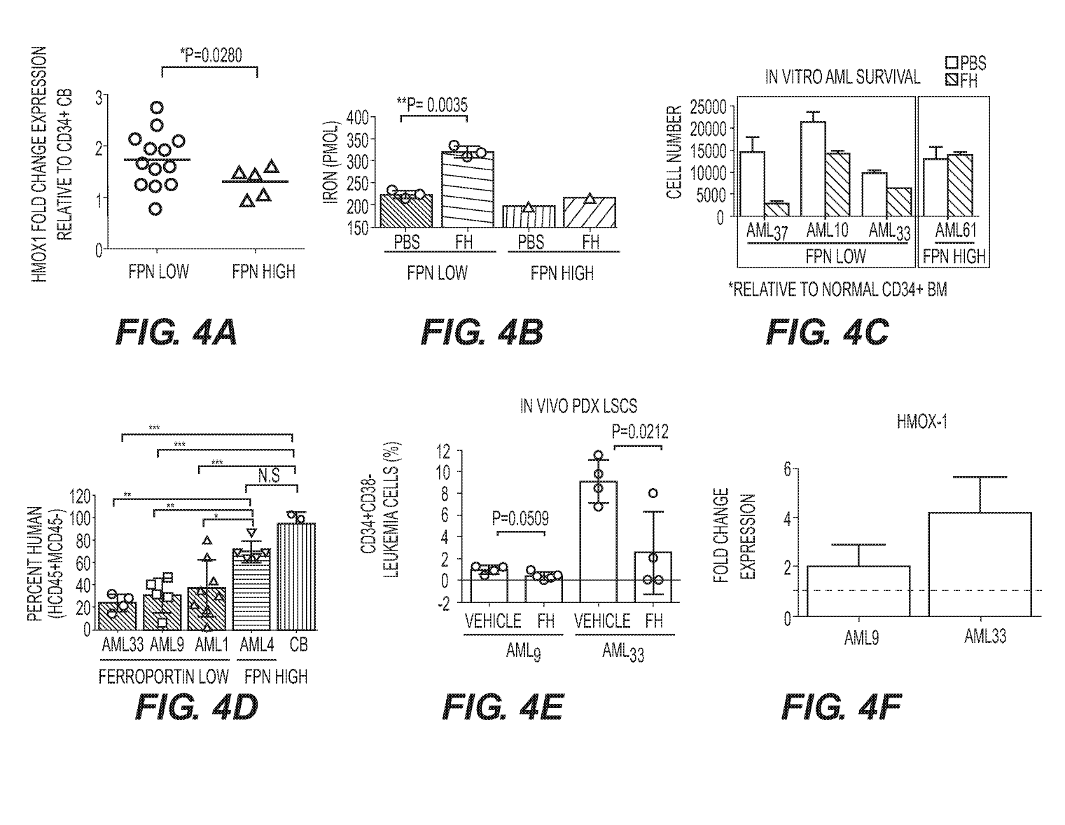

Samples with low ferroportin had higher levels of Heme Oxygenase-1 (HMOX-1, upregulated as response to ROS) (FIG. 4, panel A), and, when treated with FH, higher accumulation of iron (FIG. 4, panel B) and decreased survival in vitro (FIG. 4, panel C). In addition, treatment with FH resulted in a 2 to 3-fold decrease in AML-tumor burden after 4 weeks of treatment (1.5 mg/kg 3× a week) (FIG. 4, panel D) and also LSC (FIG. 4, panel E). This suggests that aberrant iron metabolism with decreased ferroportin may be an important hallmark of LSC biology. Increased cellular iron is associated with formation of ROS, which induces cell differentiation and at higher levels cell death (Valko et al. 2007). Therefore, LSCs may be particularly sensitive to drugs that increase iron and ROS, FH being a prime candidate as it increases both.

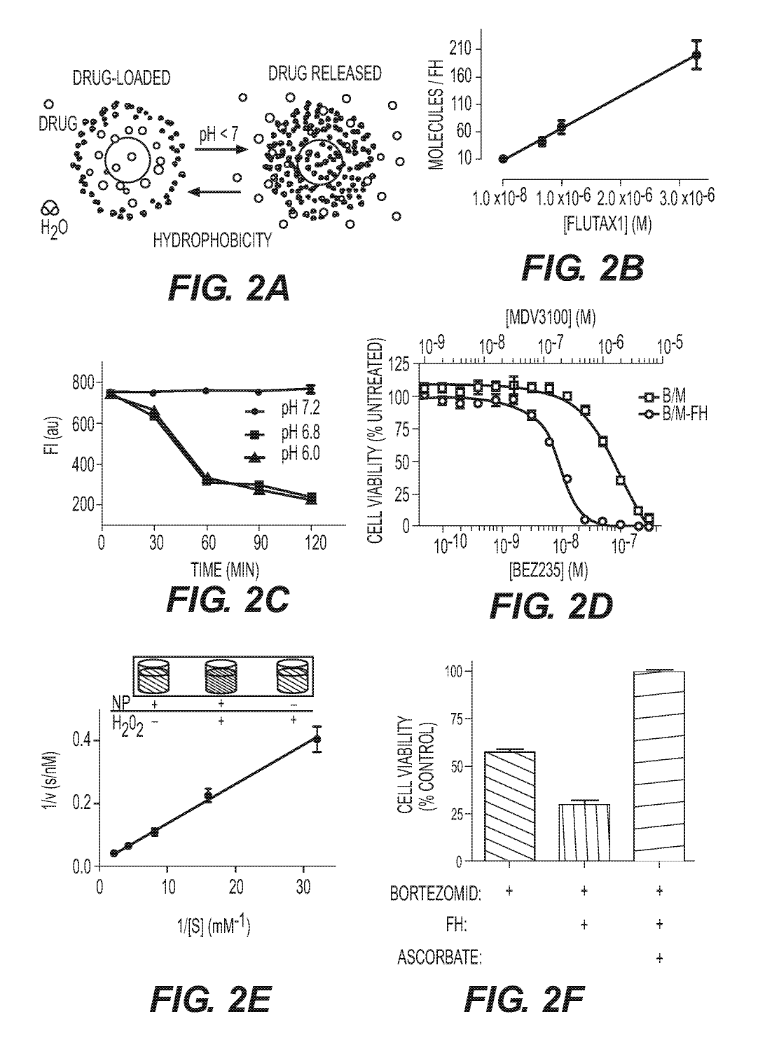

The results of these studies suggest that aberrant iron metabolism with decreased ferroportin may be an important hallmark of LSC biology. Increased cellular iron is associated with formation of ROS, which induces cell differentiation and at higher levels, cell death (Valko et al., Int. J. Biochem. Cell Biol. (2007). 39(1):44-84). These results suggest that LSCs may be particularly sensitive to drugs that increase iron and ROS. Feraheme (FH) or ferumoxytol is a clinical approved iron oxide nanoparticle for the therapy of iron deficiency anemia. It has a diameter of ca. 30 nm and a carboxy-dextran coating, which can be loaded with drugs, facilitated through weak electrostatic interactions (FIG. 2A).

### Example 2

### FH Acts Synergistically with Loaded ROS-Inducing Compounds to Kill AML Cancer Cells

Given that FH alone was able to selectively target LSCs for apoptosis, it was of interest to see whether loading of FH nanoparticles with ROS-inducing compounds could further enhance the killing of LSCs in vitro.

FH is a clinical approved iron oxide nanoparticle for the therapy of iron deficiency anemia. It has a diameter of ca. 30 nm and a carboxy-dextran coating, which can be loaded with drugs, facilitated through weak electrostatic interactions (FIG. 2, panel A). This phenomenon was verified, using fluorescent drugs such as doxorubicin or the fluorescent analog of Taxol (Flutaxl) (FIG. 2, panel B). Drug-loaded FH preparations released their cargo upon mild acidification of the medium, reminiscent of the environment in a tumor due to the upregulated aerobic glycolysis or within late endosomes. Doxorubicin-loaded FH released ˜50% of its payload within 45 minutes after encountering slightly acidic pH (FIG. 2, panel C). This approach allowed for improved drug delivery and efficacy compared to free drugs (FIG. 2, panel D). FH's iron oxide core consists of Fe(II) and Fe(III) atoms (magnetite), which possess an intrinsic peroxidase-like activity, capable of oxidizing organic substrates in a dose dependent manner (FIG. 2, panel E). Consequently, FH was able to improve or augment the efficacy of drugs, such as bortezomib, a proteasome inhibitor, which increases oxidative stress through generation of ROS. The effects of FH/bortezomib were shown to be prevented by the ROS scavenger, ascorbate (FIG. 2, panel F).

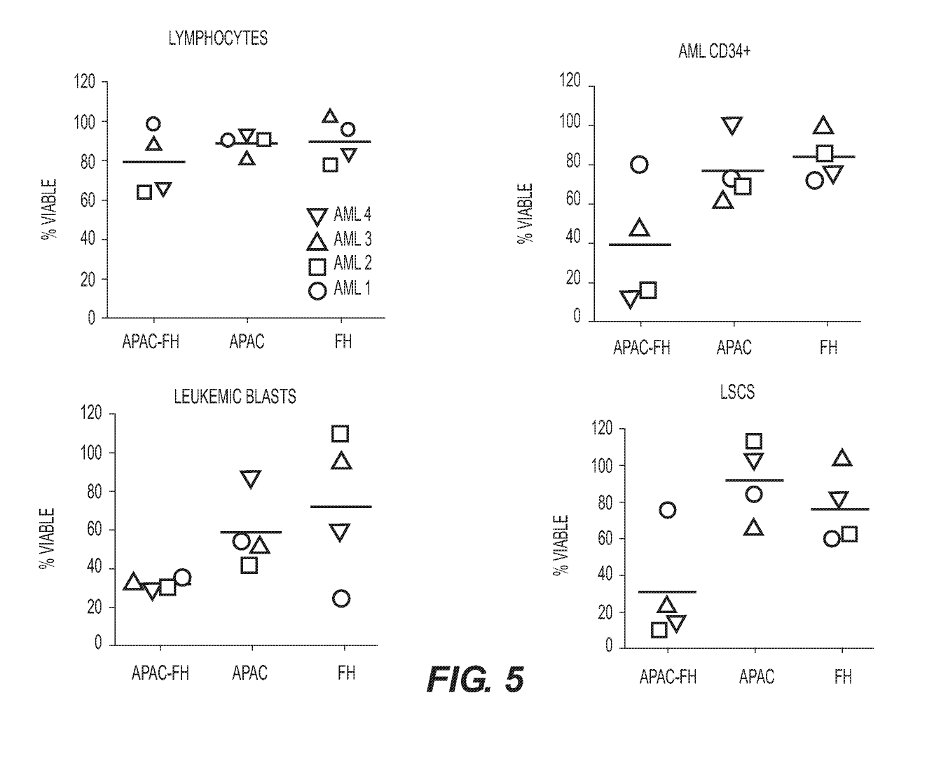

FIG. 5 shows the in vitro viability of lymphocytes (Panel A), AML CD34+ (Panel B), leukemic blasts (Panel C) or LSCs (Panel D) from four AML patient samples (AML1-AML4) after treatment with FH nanoparticles loaded with A-PAC, a natural ROS-inducing compound. As compared to control treatments with A-PAC only or FH only, FH nanoparticles loaded with A-PAC produced a synergistic effect in selectively killing leukemic cells from AML patients.

### Example 3

### FH-Meditated Drug Delivery in Other Cancers

Studies in breast cancer described a “low intracellular iron” phenotype, characterized by high ferroportin, which was associated with good prognosis. In contrast, the “high intracellular iron” phenotype, characterized by low ferroportin and high hepcidin, was associated with a poor prognosis (Pinnix et al. (2010), Sci. Transl. Med., 2(43): 43ra56). Recently, a similar observation was made in prostate cancer (Tesfay et al. (2015) Cancer Res., 75(11):2254-63. To extend the analysis to prostate cancer, LNCaP prostate cancer xenografts were treated with FH-loaded bortezomib (a ROS-inducing drug).

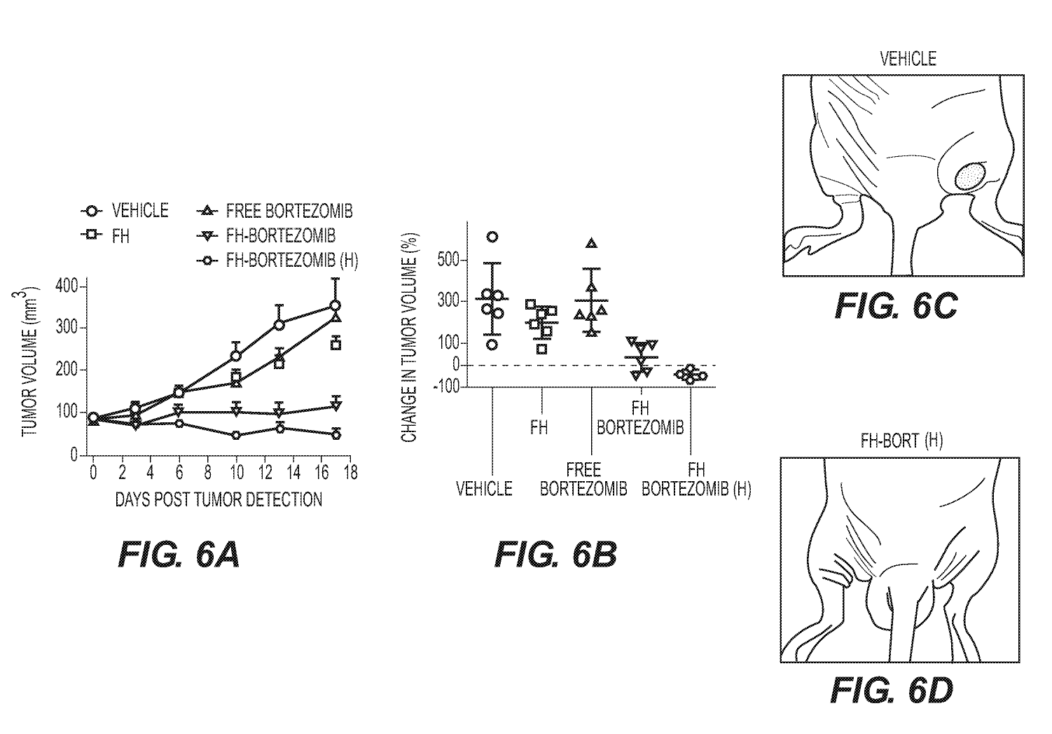

As shown in FIG. 6, significant (p=0.002) dose-dependent therapeutic effects were observed in LNCaP xenografts treated with FH-bortezomib and FH alone, but not bortezomib alone or vehicle (panel a). FH alone was able to decrease tumor volume by about 33% (panel b). FH was found to significantly enhance the efficacy of bortezomib (panels a and b). Free bortezomib was not effective, but when loaded into FH a significant therapy effect was achieved as shown in (panels a and b). Importantly, FH alone also exerted an effect, decreasing tumor volume. Each of the photographs in panel c and panel d depicts a representative animal treated with vehicle (panel c) or bortezomib-loaded FH (1.0 mM) (panel d).

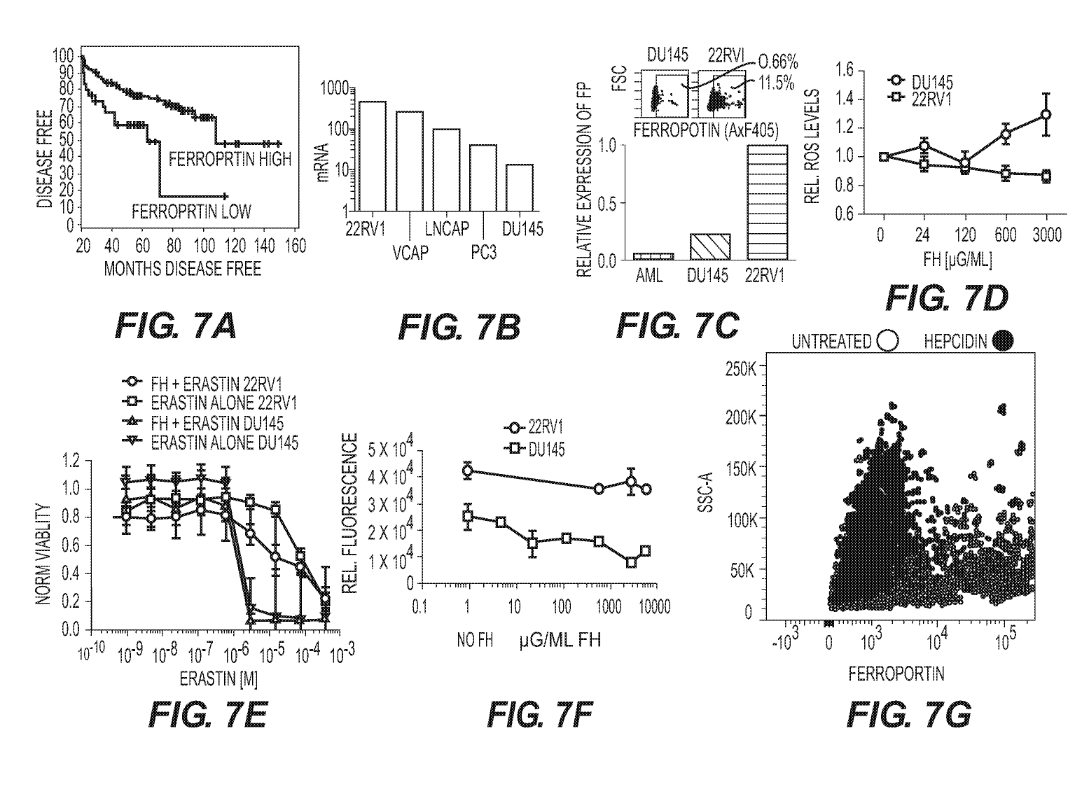

As a followup, eight different genomic datasets available for prostate cancer on cBioPortal (www.cbioportal.org) containing whole genome data from 1229 prostate cancer patients were mined for analysis. In this case, a “high intracellular iron” phenotype of PC characterized by low levels of ferroportin and significantly decreased survival was found (FIG. 7, panel A). In addition, analysis of the Cancer Genome Atlas (http://cancergenome.nih.gov) for ferroportin expression in five prostate cancer cell lines (22Rv1, VCAP, LNCaP, PC3, and DU145) revealed differences in ferroportin expression levels (FIG. 7, panel B). FACS and Western Blotting confirmed higher expression of FPN in 22RV1 compared to DU145 (FIG. 7, panel C). Treatment of 22RV1 and DU145 with FH showed that the lower ferroportin expression level in DU145 supported the largest increase in ROS at increasing FH doses (FIG. 7, panel D). Treatment of 22RV1 and DU145 with increasing amounts of erastin alone or together with constant levels of FH showed that FH improved therapy efficacy. In 22RV1 cells with higher FPN higher levels of erastin were required compared to DU145 cells with low FPN (FIG. 7, panel E). Similarly, FH alone was more efficient in the DU145 cells with lower FPN than in the 22RV1 ells with higher FPN (FIG. 7, panel F). Therapy with hepcidin also lowered FPN on the cell surface (FIG. 7, panel G).

The data above establish that FH alone has a potent anti-cancer effect in vivo, which is applicable to solid cancers, as well as leukemias. As such, treatment of cancer with FH alone or FH-loaded drugs provides a form of “oxidative ferrotherapy” in which FH can selectively exhaust the antioxidant capacity of LSCs and other cancer cells while sparing normal cells with higher ferroportin activity. Although not wishing to be bound by theory, it appears that: (1) FH increases redox active iron in LSCs and other cancer cells, generating increased levels of iron-catalyzed ROS and inducing cell death similar to ferroptosis; and (2) that FH-loaded pro-oxidants can enhance cell death of LSCs and other cancer cells through a synergistic relationship between FH and drugs, leading to robust cell death.

The foregoing descriptions of specific embodiments of the present application have been presented for purposes of illustration and description. They are not intended to be exhaustive or to limit the application and method of use to the precise forms disclosed. Obviously many modifications and variations are possible in light of the above teaching. It is understood that various omissions or substitutions of equivalents are contemplated as circumstance may suggest or render expedient, but is intended to cover the application or implementation without departing from the spirit or scope of the present application. Indeed, various modifications of the described modes for carrying out the invention that are obvious to those skilled in cellular biology, cancer cell biology, biochemistry, chemistry, organic synthesis, imaging diagnostics or related fields are intended to be within the scope of the present application.

It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. A method of treating cancer in a subject in need thereof, comprising the step of:
administering to a subject with leukemia an effective amount of a pharmaceutical composition comprising a naked metal oxide nanoparticle,
wherein the composition is not loaded with an active agent.

2. The method of claim 1, wherein the nanoparticle further comprises a polymeric coating over a metal oxide core.

3. A method of treating cancer in a subject in need thereof, comprising the step of:
administering to the subject an effective amount of a pharmaceutical composition comprising a metal oxide nanoparticle comprising a polymeric coating over a metal oxide core,
wherein the nanoparticle is loaded with a reactive oxygen species (ROS)-inducing agent.

4. The method of claim 3, wherein the reactive oxygen species (ROS) generating agent selected from the group consisting of A-type proanthocyanidins (A-PACs), erastin, bortezomib, parthenolide, elesclomol, and a combination of any two or more thereof.

5. The method of claim 1, wherein the metal oxide nanoparticle comprises non-stoichiometric magnetite.

6. The method of claim 5, wherein the metal oxide nanoparticle is coated with polyglucose sorbitol carboxymethylether or carboxymethyl dextran.

7. The method of claim 5, wherein the metal oxide nanoparticle comprises ferumoxytol.

8. The method of claim 1, wherein the cancer is a leukemia selected from the group consisting of acute myelogenous leukemia, chronic myelogenous leukemia, acute lymphoblastic leukemia, chronic lymphocytic leukemia, hairy cell leukemia, T-cell prolymphocytic leukemia, large granular lymphocytic leukemia, and adult T-cell leukemia.

9. The method of claim 8, wherein the leukemia is acute myelogenous leukemia (AML).

10. The method of claim 3, wherein the composition is not loaded with a chemotherapeutic agent.

11. The method of claim 1, wherein the nanoparticle further comprises a polymeric coating over a metal oxide core.

12. (canceled)

13. The method of claim 3, wherein the ROS-inducing agent is selected from the group consisting of A-type proanthocyanidins (A-PACs), erastin, bortezomib, parthenolide, elesclomol, and a combination of any two or more thereof.

14. The method of claim 3, wherein the metal oxide nanoparticle is an iron oxide nanoparticle.

15. The method of claim 14, wherein the iron oxide nanoparticle comprises non-stoichiometric magnetite (superparamagnetic iron oxide).

16. The method of claim 15, wherein the iron oxide nanoparticle is coated with polyglucose sorbitol carboxymethylether or carboxymethyl dextran.

17. The method of claim 15, wherein the iron oxide nanoparticle comprises ferumoxytol.

18. The method of claim 3, wherein the cancer is a leukemia selected from the group consisting of acute myelogenous leukemia, chronic myelogenous leukemia, acute lymphoblastic leukemia, chronic lymphocytic leukemia, hairy cell leukemia, T-cell prolymphocytic leukemia, large granular lymphocytic leukemia, and adult T-cell leukemia

19. The method of claim 3, wherein the cancer is a solid cancer.

20. A pharmaceutical composition for treating cancer, wherein the pharmaceutical composition comprises
(1) a nanoparticle comprising a polymeric coating over a metal oxide core, wherein the nanoparticle is loaded with a ROS-generating agent selected from the group consisting of A-PACs, erastin, bortezomib, parthenolide, and elesclomol; and
(2) a pharmaceutically acceptable carrier.

21. The pharmaceutical composition of claim 20, wherein the metal oxide core comprises non-stoichiometric magnetite.
